(19) United States
(12) Patent Application Publication
(10) Pub. No.: US 2014/0296601 A1
(43) Pub. Date: Oct. 2, 2014

(54) ISOMERIZATION PROCESS WITH MTW CATALYST

(71) Applicant: UOP LLC, Des Plaines, IL (US)

(72) Inventors: Neelesh Rane, Schaumburg, IL (US); Paula Bogdan, Mount Prospect, IL (US); Veronica G. Deak, Chicago, IL (US); Patrick Whitchurch, Sleepy Hollow, IL (US); Karl Z. Steigleder, Glen Ellyn, IL (US); Marlyn A. Hamborg, Downers Grove, IL (US); Wharton Sinkler, Des Plaines, IL (US); Steven A. Bradley, Arlington Heights, IL (US)

(73) Assignee: UOP LLC, Des Plaines, IL (US)

(21) Appl. No.: 13/853,400

(22) Filed: Mar. 29, 2013

(51) Int. Cl. C07C 5/27; B01J 29/74

(52) U.S. Cl. CPC C07C 5/2737; B01J 29/7469
USPC 585/481; 502/66

(57) ABSTRACT

An extruded C₈ alkylaromatic isomerization catalyst is described. The catalyst has an average pore diameter in a range of about 110 Å to about 155 Å measured by BJH adsorption method and a pore volume less than about 0.62 cc/g measured by N₂ porosimetry. A process for isomerizing a non-equilibrium C₈ aromatic feed to provide an isomerized product is also described.

### BACKGROUND OF THE INVENTION

The xylenes, such as para-xylene, meta-xylene and ortho-xylene, can be important intermediates that find wide and varied application in chemical syntheses. Para-xylene upon oxidation yields terephthalic acid which is used in the manufacture of synthetic textile fibers and resins. Meta-xylene can be used in the manufacture of plasticizers, azo dyes, wood preservers, etc. Ortho-xylene is a feedstock for phthalic anhydride production.

The proportions of xylene isomers from catalytic reforming or other sources generally do not match their demand as chemical intermediates. In addition, the mixture also includes ethylbenzene, which can be difficult to separate or to convert. Typically, para-xylene is a major chemical intermediate with significant demand, but it amounts to only about 20-25% of a typical C₈ aromatic stream. The adjustment of an isomer ratio to demand can be effected by combining xylene-isomer recovery, such as adsorption for para-xylene recovery, with isomerization to yield an additional quantity of the desired isomer. Typically, isomerization converts a non-equilibrium mixture of the xylene isomers that is lean in the desired xylene isomer to a mixture approaching equilibrium concentrations.

Various catalysts and processes have been developed to effect xylene isomerization. In selecting an appropriate technology, it is desirable to run the isomerization process as close to equilibrium as practical in order to maximize the para-xylene yield. However, there is a greater cyclic C₈ loss due to side reactions associated with such operation. Often, the approach to equilibrium that is used is an optimized compromise between high C₈ cyclic loss at high conversion (i.e., very close approach to equilibrium) and high utility costs due to the large recycle rate of unconverted C₈ aromatic. Thus, catalysts can be evaluated on the basis of a favorable balance of activity, selectivity, and stability.

Catalysts that can isomerize ethylbenzene to xylenes while minimizing C₈ ring loss would be beneficial.

### SUMMARY OF THE INVENTION

One aspect of the invention is an extruded C₈ alkylaromatic isomerization catalyst. In one embodiment, the catalyst comprises: about 1 to about 20% by weight of an MTW zeolite; about 80 to about 99% by weight of a binder comprising an alumina; about 0.01 to about 2.00% by weight of a Group VIII metal calculated on an elemental basis; wherein the weight percents of the MTW zeolite, the binder, and the Group VIII metal, are based on a weight of the extruded catalyst, wherein the catalyst has an average pore diameter in a range of about 110 Å to about 155 Å measured by BJH adsorption method and a pore volume less than about 0.62 cc/g measured by N₂ porosimetry.

Another aspect of the invention is a process for isomerizing a non-equilibrium C₈ aromatic feed to provide an isomerized product. In one embodiment, the process involves contacting the non-equilibrium C₈ aromatic feed with the extruded C₈ alkylaromatic isomerization catalyst described above.

### BRIEF DESCRIPTION OF THE DRAWINGS

FIG. 1 is a graph comparing the pore width measured by Hg porosimetry of one embodiment of the catalyst of the invention with a prior art catalyst.

FIG. 2 is a graph comparing the pore width measured by N₂ porosimetry of one embodiment of the catalyst of the invention with a prior art catalyst.

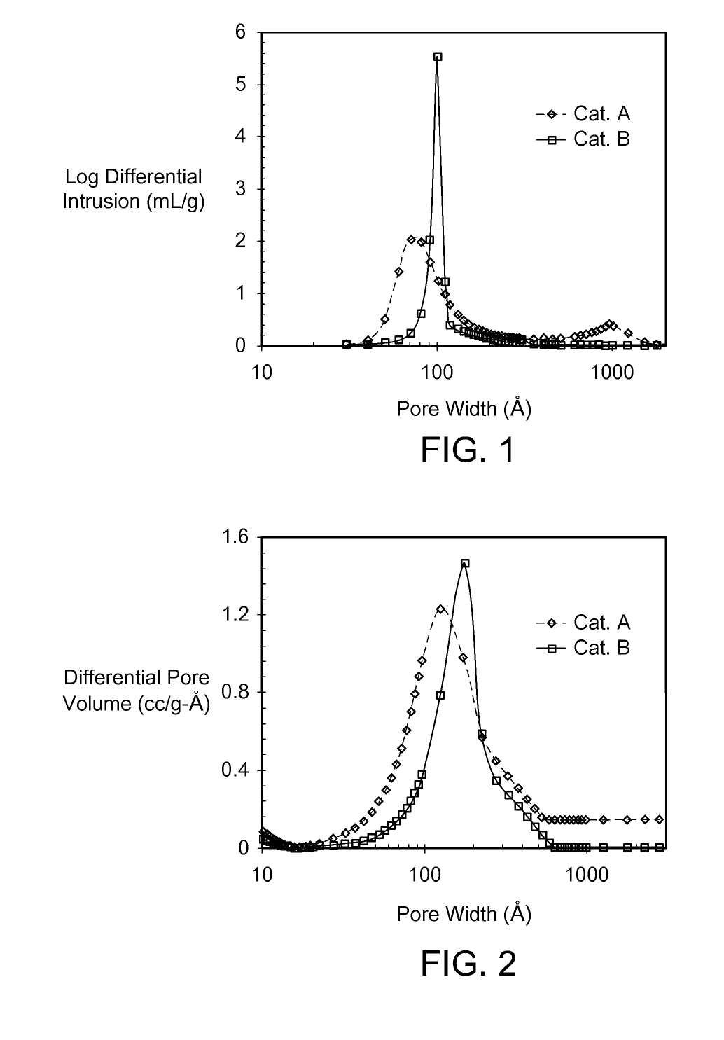

### DETAILED DESCRIPTION OF THE INVENTION

Generally, a refinery or a petrochemical production facility can include an aromatics production facility or an aromatics complex, particularly a C₈ aromatics complex that purifies a reformate to extract one or more xylene isomers, such as para-xylene or meta-xylene. Such an aromatics complex for extracting para-xylene is disclosed in U.S. Pat. No. 6,740,788. A feedstock to an aromatics complex can include an isomerizable aromatic hydrocarbon of the general formula C₆H₍₆₋ₙ₎Rₙ, where n is an integer from 2 to 5 and R is CH₃, C₂H₅, C₃H₇, or C₄H₉, in any combination and including all the isomers thereof. Suitable aromatic hydrocarbons may include ortho-xylene, meta-xylene, para-xylene, ethylbenzene, ethyltoluene, tri-methylbenzene, di-ethylbenzene, tri-ethylbenzene, methylpropylbenzene, ethylpropylbenzene, di-isopropylbenzene, or a mixture thereof.

An aromatics complex can include a xylene isomer separation zone, such as a para-xylene separation zone, and a C₈ aromatic isomerization zone. The C₈ aromatic isomerization zone can receive a stream depleted of at least one xylene isomer, such as para-xylene or meta-xylene. The C₈ aromatic isomerization zone can reestablish the equilibrium concentration of xylene isomers and convert other compounds, such as ethylbenzene, into xylenes. Typically, such a zone can increase the amount of a xylene isomer, such as para-xylene, and the product from that C₈ aromatic isomerization zone can be recycled to the xylene isomer separation zone to recover more of the desired isomer.

One exemplary application of the catalyst disclosed herein is the isomerization of a C₈ aromatic mixture containing ethylbenzene and xylenes. Generally, the mixture has an ethylbenzene content of about 1 to about 50%, by weight, an ortho-xylene content of up to about 35%, by weight, a meta-xylene content of about 20 to about 95%, by weight, and a para-xylene content of up to about 30%, by weight. The aforementioned C₈ aromatics are a non-equilibrium mixture, i.e., at least one C₈ aromatic isomer is present in a concentration that differs substantially from the equilibrium concentration at isomerization conditions. Usually the non-equilibrium mixture is prepared by removal of para-, ortho- and/or meta-xylene from a fresh C₈ aromatics mixture obtained from an aromatic production process.

Accordingly, a C₈ aromatic hydrocarbon feed mixture, preferably in admixture with hydrogen, can be contacted with a catalyst hereinafter described in a C₈ aromatic hydrocarbon isomerization zone. Contacting may be effected using the catalyst in a fixed bed system, a moving bed system, a fluidized bed system, or in a batch operation. Preferably, a fixed bed system is utilized. In this system, a hydrogen-rich gas and the feed mixture are preheated by any suitable heating means to the desired reaction temperature and then passed into a C₈ aromatic isomerization zone containing a fixed bed of catalyst. The conversion zone may be one or more separate reactors with suitable means there between to ensure that the desired isomerization temperature is maintained at the entrance of each zone. The reactants may be contacted with the catalyst bed in either upward-, downward-, or radial-flow fashion, and the reactants may be in the liquid phase, a mixed liquid-vapor phase, or a vapor phase when contacted with the catalyst.

The feed mixture, preferably a non-equilibrium mixture of C₈ aromatics, may be contacted with the isomerization catalyst at suitable C₈ isomerization conditions. Generally, such conditions include a temperature ranging from about 0° C. to about 600° C. or more, preferably about 300° C. to about 500° C. Generally, the pressure is from about 100 kPa to about 10,000 kPa absolute, preferably less than about 5,000 kPa. Sufficient catalyst may be contained in the isomerization zone to provide a liquid hourly space velocity with respect to the hydrocarbon feed mixture of from about 0.1 to about 30 hr⁻¹, and preferably about 0.5 to about 10 hr⁻¹. The hydrocarbon feed mixture can be reacted in admixture with hydrogen at a hydrogen/hydrocarbon mole ratio of about 0.5:1 to about 25:1 or more. Other inert diluents such as nitrogen, argon and light hydrocarbons may be present.

The reaction can isomerize xylenes while reacting ethylbenzene to form a xylene mixture via conversion to and reconversion from naphthenes. Thus, the yield of xylenes in the product may be enhanced by forming xylenes from ethylbenzene. Typically, the loss of C₈ aromatics through the reaction is low, generally less than about 4%, by mole, preferably no more than about 3.5%, by mole, and most preferably less than about 3%, by mole, per pass of C₈ aromatics in the feed to the reactor.

Any effective recovery scheme may be used to recover an isomerized product from the effluent of the reactors. Typically, the liquid product is fractionated to remove light and/or heavy byproducts to obtain the isomerized product. Heavy byproducts can include aromatic C₁₀ compounds such as dimethylethylbenzene. In some instances, certain product species such as ortho-xylene or dimethylethylbenzene may be recovered from the isomerized product by selective fractionation. The product from isomerization of C₈ aromatics usually is processed to selectively recover the para-xylene isomer, optionally by crystallization. Selective adsorption can be accomplished by using crystalline aluminosilicates according to U.S. Pat. No. 3,201,491.

A catalyst of the C₈ aromatic isomerization zone can include at least one MTW zeolitic molecular sieve, also characterized as “low silica ZSM-12” and can include molecular sieves with a silica to alumina ratio less than about 45, preferably from about 20 to about 40. Preferably, the MTW zeolite is substantially mordenite-free, which generally means an MTW component containing less than about 20%, by weight, mordenite impurity, or less than about 10%, by weight, or less than about 5%, by weight, mordenite.

The preparation of an MTW zeolite by crystallizing a mixture including an alumina source, a silica source and a templating agent is known. U.S. Pat. No. 3,832,449 discloses an MTW zeolite using tetraalkylammonium cations. U.S. Pat. Nos. 4,452,769 and 4,537,758 disclose a methyltriethylammonium cation to prepare a highly siliceous MTW zeolite. U.S. Pat. No. 6,652,832 uses an N,N-dimethylhexamethyleneimine cation as a template to produce low silica-to-alumina ratio MTW zeolite without MFI impurities. Preferably high purity crystals are used as seeds for subsequent batches.

The MTW zeolite is preferably composited with a binder for convenient formation of particles. The proportion of zeolite in the catalyst is about 1 to about 90% by weight, or about 1 to about 20% by weight, or about 5 to about 10% by weight. Generally, it is desirable for the MTW zeolite to contain about 0.3 to about 0.5% by weight, Na₂O and about 0.3 to about 0.5% by weight K₂O. On an elemental basis, the MTW zeolite can contain about 4,000 to 8,000 ppm by weight of at least one alkali metal, preferably sodium and/or potassium. Typically, the MTW zeolite can contain about 2,000 to about 4,000 ppm by weight sodium, and about 2,000 to about 4,000 ppm by weight potassium calculated on an elemental basis. Also, in one exemplary embodiment it is desirable for the molar ratio of silica to alumina to be about 36:1, and the molar ratio of (Na+K)/Al to be about 0.2 to about 0.3.

Generally, the zeolite is combined with a refractory inorganic oxide binder. The binder should be a porous, adsorptive support having a surface area of about 25 to about 500 m²/g, preferably about 100 to about 400 m²/g. Desirably, the inorganic oxide is an alumina, such as a gamma-alumina. Such a gamma-alumina can be derived from a boehmite or a pseudoboehmite alumina (hereinafter collectively may be referred to as “boehmite alumina”). The boehmite alumina can be compounded with the zeolite and extruded. During oxidation (or calcination), the boehmite alumina may be converted into gamma-alumina. Suitable boehmite alumina utilized as starting material includes TH and TM type sold by SASOL. The SASOL boehmite alumina can be blended with other boehmite alumina, such as V-251 available from UOP LLC. If a blend is used, it is desirable that at least about 50% of the catalyst is SASOL TH and TM type alumina, or at least about 60%, or at least about 70%, or at least about 80%, or at least about 85%, or at least about 90%, or at least about 95%.

Although not wishing to be bound by theory, one of the desired boehmite aluminas, TM-100, appears to be like fine rods, which is expected to give a more interconnected pore system. TM-100 also has uniform particles compared to the non-uniform heterogeneous morphology of V-251.

Generally, the catalyst can have about 1 to about 99% by weight of the alumina binder, or about 90 to about 99% by weight, or about 90 to about 95% by weight.

One shape for the support or catalyst can be an extrudate. Generally, the extrusion initially involves mixing of the molecular sieve with optionally the binder and a suitable peptizing agent to form a homogeneous dough or thick paste having the correct moisture content to allow for the formation of extrudates with acceptable integrity to withstand direct calcination. Extrudability may be determined from an analysis of the moisture content of the dough, with a moisture content in the range of from about 30 to about 70% by weight being desirable. Methocel™ cellulose ether (available from Dow Chemical Co.) and Solka-floc® 40 powdered cellulose (available from International Fiber Corp.) may be used as aids for extrusion process. The dough may then be extruded through a die pierced with multiple holes and the spaghetti-shaped extrudate can be cut to form particles in accordance with known techniques. A multitude of different extrudate shapes is possible, including a cylinder, cloverleaf, dumbbell, and symmetrical and asymmetrical polylobates. Furthermore, the dough or extrudates may be shaped to any desired form, such as a sphere, by, e.g., marumerization that can entail one or more moving plates or compressing the dough or extrudate into molds.

Alternatively, support or catalyst pellets can be formed into spherical particles by accretion methods. Such a method can entail adding liquid to a powder mixture of zeolite and binder in a rotating pan or conical vessel having a rotating auger.

Generally, preparation of alumina-bound spheres involves dropping a mixture of molecular sieve, alsol, and gelling agent into an oil bath maintained at elevated temperatures. Examples of gelling agents that may be used in this process include hexamethylene tetraamine, urea, and mixtures thereof. The gelling agents can release ammonia at the elevated temperatures which sets or converts the hydrosol spheres into hydrogel spheres. The spheres may then be withdrawn from the oil bath and typically subjected to specific aging treatments in oil and an ammonia solution to further improve their physical characteristics. One exemplary oil dropping method is disclosed in U.S. Pat. No. 2,620,314.

Generally, the subsequent drying, calcining, and optional washing steps can be done before and/or after impregnation with one or more components, such as metal. Preferably after formation of the binder and zeolite into a support, the support can be dried at a temperature of about 50° C. to about 320° C., or about 100 to about 200° C. for a period of about 1 to about 24 hours or more. Next, the support is usually calcined or oxidized at a temperature of 50° C. to about 700° C., desirably about 540° C. to about 650° C. for a period of about 1 to about 20 hours, or about 1 to about 1.5 hours in an air atmosphere until the metallic compounds, if present, are converted substantially to the oxide form, and substantially all the alumina binder is converted to gamma-alumina. If desired, the optional halogen component may be adjusted by including a halogen or halogen-containing compound in the air atmosphere. The various heat treating steps may be conducted multiple times such as before and after addition of components, such as one or more metals, to the support via impregnation as is well known in the art. Steam may be present in the heat treating atmospheres during these steps. During calcination and/or other heat treatments to the catalyst, the pore size distribution of the alumina binder can be shifted to larger diameter pores. Thus, calcining the catalyst can increase the average pore size of the catalyst.

Optionally, the catalyst can be washed. Typically, the catalyst can be washed with a solution of ammonium nitrate or ammonium hydroxide, preferably ammonium hydroxide. Generally, the wash is conducted at a temperature of about 50° C. to about 150° C. for about 1 to about 10 hours. In one desired embodiment, no wash is conducted to provide an elevated level of at least one alkali metal. Generally, a wash of ammonium nitrate can lower the amount of alkali metal in the catalyst, particularly the zeolite. Exemplary catalysts without a wash are depicted in US Pub. No. 2005/0143615 A1. Preferably, no wash or a wash of ammonium hydroxide is conducted to allow much of the existing alkali metal to remain on the catalyst. It should be understood, however, if the zeolite and/or binder, particularly the zeolite, has an elevated alkali metal content, then an ammonium nitrate wash can be conducted that allows some alkali metal at a desired level to remain on the zeolite and/or binder.

In some exemplary embodiments, after drying, calcining, and optionally washing, one or more components can be impregnated on the support. The catalyst may also include a Group VIII (IUPAC 8-10) metal, including one or more of platinum, palladium, rhodium, ruthenium, osmium, and iridium. The preferred Group VIII metal is platinum. The Group VIII metal component may exist within the final catalyst as a compound such as an oxide, sulfide, halide, or oxysulfide, or as an elemental metal or in combination with one or more other ingredients of the catalyst. Desirably, the Group VIII metal component exists in a reduced state. This component may be present in the final catalyst in any amount which is catalytically effective. Generally, the final catalyst includes about 0.01 to about 2%, desirably about 0.05 to about 1%, and optimally about 0.25 to about 0.5% by weight calculated on an elemental basis of the Group VIII metal, preferably platinum.

The Group VIII metal component may be incorporated into the catalyst in any suitable manner. One method of preparing the catalyst involves the utilization of a water-soluble, decomposable compound of a Group VIII metal to impregnate the calcined sieve-binder composite. Alternatively, a Group VIII metal compound may be added at the time of compositing the sieve component and binder. Complexes of Group VIII metals that may be employed in impregnating solutions, co-extruded with the sieve and binder, or added by other known methods can include chloroplatinic acid, chloropalladic acid, ammonium chloroplatinate, bromoplatinic acid, platinum trichloride, platinum tetrachloride hydrate, platinum dichlorocarbonyl dichloride, tetraamine platinic chloride, dinitrodiaminoplatinum, sodium tetranitroplatinate (II), palladium chloride, palladium nitrate, palladium sulfate, diaminepalladium (II) hydroxide, and tetraminepalladium (II) chloride.

A Group IVA (IUPAC 14) metal component may also be incorporated into the catalyst. Of the Group IVA (IUPAC 14) metals, germanium and tin are preferred and tin is especially preferred. This component may be present as an elemental metal, as a chemical compound such as the oxide, sulfide, halide, or oxychloride, or as a physical or chemical combination with the porous carrier material and/or other components of the catalyst. Preferably, a substantial portion of the Group IVA (IUPAC 14) metal exists in the finished catalyst in an oxidation state above that of the elemental metal. The Group IVA (IUPAC 14) metal component optimally is utilized in an amount sufficient to result in a final catalyst containing about 0.01 to about 5%, by weight, or about 0.1 to about 2%, by weight, or about 0.3-about 0.45% by weight metal calculated on an elemental basis.

The Group IVA (IUPAC 14) metal component may be incorporated in the catalyst in any suitable manner to achieve a homogeneous dispersion, such as by co-precipitation with the porous carrier material, ion-exchange with the carrier material or impregnation of the carrier material at any stage in the preparation. One method of incorporating the Group IVA (IUPAC 14) metal component into the catalyst involves the utilization of a soluble, decomposable compound of a Group IVA (IUPAC 14) metal to impregnate and disperse the metal throughout the porous carrier material. The Group IVA (IUPAC 14) metal component can be impregnated either prior to, simultaneously with, or after the other components are added to the carrier material. Thus, the Group IVA (IUPAC 14) metal component may be added to the carrier material by commingling the latter with an aqueous solution of a suitable metal salt or soluble compound such as stannous bromide, stannous chloride, stannic chloride, stannic chloride pentahydrate; germanium oxide, germanium tetraethoxide, or germanium tetrachloride; or lead nitrate, lead acetate, or lead chlorate. The utilization of Group IVA (IUPAC 14) metal chloride compounds, such as stannic chloride, germanium tetrachloride or lead chlorate, is particularly preferred since they can facilitate the incorporation of both the metal component and at least a minor amount of the preferred halogen component in a single step. When combined with hydrogen chloride during the especially preferred alumina peptization step as described above, a homogeneous dispersion of the Group IVA (IUPAC 14) metal component can be obtained. In an alternative embodiment, organic metal compounds such as trimethyltin chloride and dimethyltin dichloride are incorporated into the catalyst during the peptization of the alumina with hydrogen chloride or nitric acid.

The catalyst may also contain other metal components as well. Such metal modifiers may include rhenium, cobalt, nickel, indium, gallium, zinc, uranium, dysprosium, thallium, or a mixture thereof. Generally, a catalytically effective amount of such a metal modifier may be incorporated into a catalyst to effect a homogeneous or stratified distribution.

The catalyst can also contain a halogen component, such as fluorine, chlorine, bromine, iodine or a mixture thereof, with chlorine being preferred. Desirably, the catalyst contains no added halogen other than that associated with other catalyst components.

The catalyst may also contain at least one alkali metal with a total alkali metal content of the catalyst of at least about 100 ppm, by weight, calculated on an elemental basis. The alkali metal can be lithium, sodium, potassium, rubidium, cesium, francium, or a combination thereof. Preferred alkali metals can include sodium and potassium. Desirably, the catalyst contains no added alkali metal other than that associated with the zeolite and/or binder. Generally, the total alkali metal content of the catalyst is at least about 200 ppm, or at least about 300 ppm by weight calculated on an elemental basis. Generally, the total alkali metal content of the catalyst is no more than about 2500 ppm, or about 2000 ppm, or about 1000 ppm by weight calculated on an elemental basis. In one preferred embodiment, the catalyst can have about 300 ppm to about 2500 ppm by weight of at least one alkali metal calculated on an elemental basis. In a further embodiment, the catalyst can have about 100 ppm to less than about 1000 ppm, or about 300 to less than about 1000 ppm, or about 300 to about 700 ppm by weight of at least one alkali metal, preferably sodium and/or potassium, calculated on an elemental basis. In yet another preferred embodiment, the catalyst can have at least about 150 ppm, or about 150 to about 310 ppm by weight sodium and at least about 50 ppm, or about 50 to about 250 ppm by weight potassium, calculated on an elemental basis.

The resultant catalyst can subsequently be subjected to a substantially water-free reduction step to ensure a uniform and finely divided dispersion of the optional metallic components. The reduction may be effected in the process equipment of the aromatic complex. Substantially pure and dry hydrogen (i.e., less than about 100 vol. ppm, preferably about 20 vol. ppm, H₂O) preferably is used as the reducing agent. The reducing agent can contact the catalyst at conditions, including a temperature of about 200° C. to about 650° C. and a period of about 0.5 to about 10 hours, effective to reduce substantially all of the Group VIII metal component to the metallic state. In some cases, the resulting reduced catalyst may also be beneficially subjected to presulfiding by a known method such as with neat H₂S at room temperature to incorporate in the catalyst an amount of about 0.05 to about 1.0% by weight sulfur, calculated on an elemental basis.

The elemental analysis of the components of the zeolite and/or catalyst, such as Group VIII metal component and/or the at least one alkali metal can be determined by Inductively Coupled Plasma (ICP) analysis according to UOP Method 961-98. The elemental analysis of an alkali metal, such as sodium, in an alumina binder, can be conducted by ICP or atomic adsorption spectroscopy analysis. Regarding atomic adsorption spectroscopy analysis, sodium content can be determined according to UOP Method 410-85 and potassium content can be determined according to UOP Method 878-87.

Generally, catalysts described herein have several beneficial properties that provide isomerization of ethylbenzene while minimizing C₈ ring-loss. Although not wanting to be bound by theory, it is generally thought that the higher levels (greater than about 100 ppm by weight calculated on an elemental basis based on the weight of the catalyst) of at least one alkali metal can reduce C₈ ring loss. Thus, contacting a non-equilibrium C₈ aromatic feed with an extruded C₈ alkylaromatic isomerization catalyst can provide an isomerized product with a C₈ ring loss of no more than about 2.5, about 2.0 to about 2.5, or about 2.5.

The catalyst has an average pore diameter in a range of about 110 Å to about 155 Å measured by BJH (Barret-Joyner-Halenda) adsorption method according to UOP Method 964-98. It has a pore volume less than about 0.62 cc/g measured by N₂ porosimetry. In some embodiments, the catalyst has a porosity of less than about 75% measured by Hg porosimetry, or less than 70%. In some embodiments, it has a median pore diameter greater than about 100 Å measured by Hg porosimetry.

One of the embodiments involving MTW/TM-100 (5%/95%) catalyst had lower pore volume and higher pore diameter as measured by N₂ porosimetry compared with MTW/V-251 (5%/95%). The MTW/TM-100 catalysts had a narrow pore distribution as measured by Hg porosimetry according to UOP Method 578-84. There was a similar Pt cluster size and high Pt dispersion over both TM-100 and V-251 based catalysts. There was no difference in the Pt reproducibility of the TM-100 compared with the V-251.

All the UOP methods, such as UOP 410-85, UOP 878-87, UOP 578-84, UOP 964-98 and UOP 961-98, discussed herein can be obtained through ASTM International, 100 Barr Harbor Drive, West Conshohocken, Pa., USA.

### EXAMPLES

The following catalysts are intended to further illustrate the subject catalyst. These illustrations of embodiment are not meant to limit the claims of this invention to the particular details of these examples. These examples are based on engineering calculations and actual operating experiments with similar processes.

The exemplary catalysts can have a commercial synthesized MTW Zeolite and an alumina source of either VERSAL-251 (V-251) sold by UOP LLC or an alumina sold under the trade designation TM-100 by SASOL. To form the extrudate supports, the alumina is usually at least partially peptized with a peptizing agent such as nitric acid. The zeolite can be mixed with the at least partially peptized alumina or may be mixed with the alumina prior to peptization. Afterwards, typically the alumina and MTW Zeolite mixture is extruded into a tri-lobe shape. That being done, the extrudate can be dried and then calcined at about 540-about 650° C. for about 60-about 240 minutes. All the supports can be impregnated with platinum with a solution of chloroplatinic acid mixed with water and HCl. Generally, the HCl is in an amount of about 2-3%, by weight, of the support, and the excess solution is evaporated. Next, the supports can be oxidized or calcined at a temperature of about 565° C. for about 60-about 120 minutes in an atmosphere of about 5-about 15 mol % of steam with a water to chloride ratio of about 50:1-about 120:1.

Generally afterwards, the supports are reduced at about 565° C. for about 120 minutes in a mixture of at least about 15 mol % hydrogen in nitrogen. That being done, the supports can be sulfided in a 10 mol % atmosphere of hydrogen sulfide in a hydrogen sulfide and hydrogen mixture at ambient conditions to obtain about 0.07%, by weight, sulfur on the support to obtain the final catalysts.

A depiction of the materials and forming method for the exemplary catalysts is provided in the table below.

| | | | | Amount |
|---|---|---|---|---|
| Catalyst | | Forming | Alumina | MTW |
| Example | Shape | Method | Source | Weight % |
| A | Trilobe | Extrusion | V-251 | 5 |
| B | Trilobe | Extrusion | TM-100 | 5 |

Catalyst A: MTW zeolite is admixed with V-251 to provide a composite of 5 mass-parts of MTW to 95 mass-parts of V-251. The composite is extruded to form pellets. The pellets are first dried and then calcined in air at 577° C. for 4 hours. The pellets are then impregnated with a solution of chloroplatinic acid with 3.0 mass-% hydrochloric acid to provide a final platinum level of 0.31 mass-% on the final catalyst. The impregnated pellets are then oxidized at 565° C. in an atmosphere of 10% steam with a water to chloride ratio of 80:1. The pellets are then reduced at about 565° C. for 2 hours, and sulfided in a 10 mol % atmosphere of hydrogen sulfide in a hydrogen sulfide and hydrogen mixture at ambient conditions to yield 0.07 mass-% sulfur on the catalyst.

Catalyst B: MTW zeolite is admixed with TM-100 to provide a composite of 5 mass-parts of MTW to 95 mass-parts of TM-100. The composite is extruded to form pellets. The pellets are first dried and then calcined in air at 577° C. for 4 hours. The pellets are then impregnated with a solution of chloroplatinic acid with 3.0 mass-% hydrochloric acid to provide a final platinum level of 0.31 mass-% on the final catalyst. The impregnated pellets are then oxidized at 565° C. in an atmosphere of 10% steam with a water to chloride ratio of 80:1. The pellets are then reduced at about 565° C. for 2 hours, and sulfided in a 10 mol % atmosphere of hydrogen sulfide in a hydrogen sulfide and hydrogen mixture at ambient conditions to yield 0.07 mass-% sulfur on the catalyst.

Several property measurements are depicted below for the reduced catalysts, before sulfiding:

| | N₂ Porosimetry | | | Hg Porosimetry | | |
|---|---|---|---|---|---|---|
| | BET SA | BJH Adsorption | Pore | | Median | Piece Density |
| Catalyst | square- | Avg. Pore | Volume | | Pore | (Volatile Free) |
| Example | meter/gram | Diameter Å | cc/g | Porosity % | Diameter Å | g/cc |
| A | 250 | 120 | 0.776 | 78.3 | 97 | 0.795 |
| B | 165 | 141 | 0.572 | 67.7 | 105 | 1.115 |

Moreover, the reduced catalysts were evaluated for components. All components are provided in percent, by weight.

| Catalyst | Pt | Cl | Na | K |
|---|---|---|---|---|
| Example | Weight % | Weight % | Weight % | Weight % |
| A | 0.31 | 0.88 | 0.028 | 0.019 |
| B | 0.325 | 0.65 | 0.026 | 0.018 |

The catalysts are sulfided and evaluated for xylene isomerization activity using a pilot plant flow reactor processing a non-equilibrium C₈ aromatic feed having the following approximate composition in percent, by weight:

Feed Composition

| Component | Weight % |
|---|---|
| Ethylbenzene | 15 |
| Para-xylene | <1 |
| Meta-xylene | 60 |
| Ortho-xylene | 25 |
| Toluene | <1 |
| Nonaromatics | <1 |

This feed is contacted with a catalyst at a pressure of about 700 kPa(g), a weight hourly space velocity (may be referred to as WHSV) of 7 hr⁻¹, and a hydrogen/hydrocarbon mole ratio of 4. The reactor temperature is about 385° C. One method of measuring xylene isomerization activity is comparing a ratio of para-xylene in product to the total xylene in product, defined as pX/X ratio, where:

pX represents moles of para-xylene in product; and X represents moles of xylene in the product.

Generally, para-xylene is a desirable C8 aromatic. A higher pX/X at a given reactor temperature can indicate a more active catalyst. Sulfided catalysts are tested in the pilot plant for activity with the following results:

| Catalyst Example | Alumina Source | Ratio pX/X |
|---|---|---|
| A | V-251 | 0.220 |
| B | TM-100 | 0.234 |

As depicted above, the pX/X ratio is compared with the catalyst alumina source. Catalysts derived from TM-100 have substantially higher pX/X ratio than catalysts derived from V-251.

In the foregoing, all temperatures are set forth uncorrected in degrees Celsius and, all parts and percentages are by weight, unless otherwise indicated.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. An extruded C₈ alkylaromatic isomerization catalyst, comprising:
about 1 to about 20% by weight of an MTW zeolite;
about 80 to about 99% by weight of a binder comprising an alumina;
about 0.01 to about 2.00% by weight of a Group VIII metal calculated on an elemental basis;
wherein the weight percents of the MTW zeolite, the binder, and the Group VIII metal, are based on a weight of the extruded catalyst, wherein the catalyst has an average pore diameter in a range of about 110 Å to about 155 Å measured by BJH adsorption method and a pore volume less than about 0.62 cc/g measured by N₂ porosimetry.

2. The catalyst of claim 1 wherein the catalyst has a porosity of less than about 75% measured by Hg porosimetry.

3. The catalyst of claim 2 wherein the porosity is less than about 70%.

4. The catalyst of claim 1 wherein the catalyst has a median pore diameter greater than about 100 Å measured by Hg porosimetry.

5. The catalyst of claim 1 wherein the pore volume is less than about 0.60 cc/g.

6. The catalyst of claim 1 wherein the catalyst is in a shape of a cylinder, a cloverleaf, a dumbbell, a symmetrical polylobate, an asymmetrical polylobate, or combinations thereof.

7. The catalyst of claim 1 wherein the extruded catalyst comprises about 100 to less than about 1000 ppm, by weight, of at least one alkali metal calculated on an elemental basis based on the weight of the extruded catalyst.

8. A process for isomerizing a non-equilibrium C₈ aromatic feed to provide an isomerized product comprising:
contacting the non-equilibrium C₈ aromatic feed with an extruded C₈ alkylaromatic isomerization catalyst, comprising:
about 1 to about 20% by weight of an MTW zeolite;
about 80 to about 99% by weight of a binder comprising an alumina;
about 0.01 to about 2.00% by weight of a Group VIII metal calculated on an elemental basis;
wherein the weight percents of the MTW zeolite, the binder, and the Group VIII metal, are based on a weight of the extruded catalyst, wherein the catalyst has an average pore diameter in a range of about 110 Å to about 155 Å measured by BJH adsorption method and a pore volume less than about 0.62 cc/g measured by N₂ porosimetry.

9. The process of claim 8 wherein the catalyst has a porosity of less than about 75% measured by Hg porosimetry.

10. The process of claim 9 wherein the porosity is less than about 70%.

11. The process of claim 8 wherein the catalyst has a median pore diameter greater than 100 Å measured by Hg porosimetry.

12. The process of claim 8 wherein a pore volume is less than about 0.60 cc/g.

13. The process of claim 8 wherein the catalyst is in a shape of a cylinder, a cloverleaf, a dumbbell, a symmetrical polylobate, an asymmetrical polylobate, or combinations thereof.

14. The process of claim 8 wherein the extruded catalyst comprises about 100 to less than about 1000 ppm, by weight, of at least one alkali metal calculated on an elemental basis based on the weight of the extruded catalyst.
